(19) United States
(12) Patent Application Publication
(10) Pub. No.: US 2015/0196494 A1
(43) Pub. Date: Jul. 16, 2015

(54) STABILIZED PICOPLATIN ORAL DOSAGE FORM

(71) Applicant: Tallikut Pharmaceuticals, Inc., San Francisco, CA (US)

(72) Inventors: Alistair J. Leigh, Seattle, WA (US); Christopher A. Procyshyn, Surrey (CA); Ernest S.Y. Wong, Superior, CO (US); Christen M. Giandomenico, Blaine, WA (US)

(21) Appl. No.: 14/668,163

(22) Filed: Mar. 25, 2015

Related U.S. Application Data
(63) Continuation of application No. 14/121,154, filed on Aug. 6, 2014, now abandoned.
Application No. 14/121,154 is a continuation of application No. 13/587,711, filed on Aug. 16, 2012, now abandoned.
Application No. 13/587,711 is a continuation of application No. 12/536,335, filed on Aug. 5, 2009, now abandoned.

(51) Int. Cl. A61K 9/28; A61K 31/555

(52) U.S. Cl. CPC A61K 9/28; A61K 31/555

(57) ABSTRACT

The invention provides an oral dosage form for the anti-cancer drug picoplatin comprising a core and a coating, the dosage form being free of redox-active, metal salts. The core of the tablet is a substantially dry powder comprising about 10 to 60 wt % picoplatin wherein the picoplatin is a particulate of less than about 10 microns average particle diameter, about 40-80 wt % of a filler comprising a substantially water-soluble, water-dispersible, or water-absorbing carbohydrate, and an effective amount of up to about 5 wt % of a lubricant. The dosage form can further include a dispersant.

### RELATED APPLICATIONS

This application is a continuation of U.S. patent application Ser. No. 12/536,335, filed Aug. 5, 2009, which is a continuation under 35 U.S.C. III (a) of International Application No. PCT/US2008/001752 filed Feb. 8, 2008 and published in English as WO 2008/097661 on Aug. 14, 2008, which claims the benefit of provisional application Ser. No. 60/889,681, filed Feb. 13, 2007 and to provisional application Ser. No. 60/889,171, filed Feb. 9, 2007, which applications and publication are incorporated herein in their entireties.

### FIELD OF THE INVENTION

The field of the invention is a stabilized oral dosage form for the anti-cancer organoplatinum drug picoplatin, processes of preparation of the oral dosage form, and methods of use of the oral dosage form in the treatment of cancer.

### BACKGROUND

Picoplatin is a new-generation organoplatinum drug that has promise for treatment of various types of malignancies, including those that have developed resistance to earlier organoplatinum drugs such as cisplatin and carboplatin. Picoplatin has shown promise in the treatment of various kinds of cancer or tumor, including small cell lung cancer, colorectal cancer, and hormone-refractory prostate cancer.

Structurally, picoplatin is:

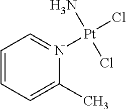

and is named cis-amminedichloro(2-methylpyridine)platinum(II), or alternatively [SP-4-3]-ammine(dichloro)(2-methylpyridine)platinum(II). The compound is a square planar complex of divalent platinum that is tetracoordinate and has three different ligand types. Two ligands are anionic, and two are neutral; therefore as the platinum in picoplatin carries a +2charge, picoplatin is itself a neutral compound and no counterions need be present. The name “picoplatin,” referring to the presence of α-picoline (2-methylpyridine) in the molecule, is the United States Adopted Name (USAN), the British Approved Name (BAN), and the international Nonproprietary Name (INN) for this material. Picoplatin is also referred to in the literature as JM473, NX473, ZD0473, and AMD473, and is disclosed in U.S. Pat. Nos. 5,665,77 6,518,428, and U.S. Ser. No. 10/276,503.

Tetracoordinate square planar platinum (II) are well known to be subject to oxidation to octahedral Pt(IV) complexes, such as with molecular chlorine. Also, it is well known that square planar platinum (II) complexes are subject to axial attack in ligand displacement reactions by various nucleophiles such as halides, amines, thio compounds, and under some conditions, water. Therefore, while picoplatin is relatively stable in pure form it can be subject to degradation under certain conditions, such as in the presence of nucleophilic molecular entities. See Advanced Inorganic Chemistry, F. Albert Cotton and Geoffrey Wilkinson, Second Revised Edition (1966) and later editions, Interscience Publishers. When administered to patients, picoplatin is believed to undergo metabolic transformation to some extent to two distinct aqua forms resulting from displacement of either of the chloride ligands. These cationic species (resulting from displacement of a chloride anion by neutral water) are reactive, and interact with cellular DNA to bring about cross-linking and eventual cell death.

Picoplatin is also known to be unstable in the presence of light. It absorbs visible light, particularly at the blue/violet end of the spectrum, which brings about photo-decomposition. It is known in the art to provide coatings for oral dosage forms that are adapted to reduce the exposure of the active ingredient to light. For example, Colorcon Inc., a manufacturer of OPADRY® coatings for pharmaceuticals, states on its website www.colorcon.com that “film coatings have the ability to-offer protection to cores that contain actives susceptible to light degradation. A film coating can give this protection by the use of an opacifying (hiding) agent such as titanium dioxide in the film coating. This has the ability to reflect light, reducing the amount of light entering, the tablet core.”

Picoplatin has previously been provided to patients in solution by intravenous (IV) administration. Picoplatin under standard conditions is a solid, and has only sparing solubility in water. The relatively low solubility of picoplatin in water (about 1 mg/mL) necessitates that substantial volumes of liquid be delivered intravenously to provide a patient with total doses in the range of 100 mg and more (i.e., at a concentration of 0.5 mg/mL, some 200 mL of liquid must be introduced by IV infusion to provide a 100 mg dose). As typical human dosages for cancer patients can be on the order of several hundred milligrams per administration, and may be repeated every few weeks, substantial volumes of liquid must be delivered to the patient for each administration of the substance by the IV route. Intravenous administration is thus undesirable due to the need for needle insertion into a vein, and the relatively prolonged periods over which the patient must be immobile to allow for infusion of the relatively large volumes of the picoplatin solutions. Picoplatin is also known to be particularly susceptible to photo-decomposition when in solution, as in an IV dosage form. Picoplatin is orally bioavailable, but its low stability in water, instability, toxicity and teratogenicity pose obstacles to the preparation of effective oral dosage forms. Therefore there is a need for effective dosage forms of picoplatin.

### SUMMARY OF THE INVENTION

The present invention provides an oral dosage form for picoplatin wherein the dosage form comprises a solid core comprising about 10 to 60 wt % particulate picoplatin wherein the picoplatin is a particulate of less than about 10 microns average particle diameter, about 40-80 wt % of a filler comprising a substantially water-soluble, water-dispersible, or water-absorbing carbohydrate, and an effective amount of up to about 5 wt % of a lubricant, and optionally a dispersant; and a continuous coating on the outer surface of the core; wherein the core and/or the coating are substantially free of redox-active metal salts. Preferably both the coating and the core are free of amounts of redox-active metals that can be deleterious to the picoplatin in vivo or in vitro (e.g., in storage).

The coating forms a protective covering for the core, both protecting the contents from environmental degradation by oxygen, light, and reactive chemicals, and protecting persons handling the dosage form from the cytotoxic picoplatin. The coating can comprise gelatin, either hard or soft; a polymer, for example hydroxypropyl methyl cellulose; a sugar, for example sucrose; or any other non-toxic, water soluble material suitable for human consumption.

The picoplatin particulate that has an average particle diameter of less than about 10 microns, preferably has an average particle diameter of less than about 7 microns, and more preferably has a particle size distribution such that about 90% of the individual particulates have a diameter of less than about 5 microns.

The core can be compacted to form a tablet core then coated to yield a tablet, molded to form a pill core then coated to form a pill, or granular, as may be used for a capsule fill. The picoplatin particulate can be a micronized material, for example as can be obtained by jet-milling, or can be a microcrystalline material, such as can be prepared by precipitation from a solvent, or can be a particulate formed by a lyophilization process, or can be formed by any combination of the three processes resulting in the desired small particle size. The picoplatin particulate material forms a portion of the core, which also includes a filler comprising a carbohydrate, a lubricant, and optionally a dispersing agent. The filler makes up about 40-80 wt % of the material composing the core of the tablet, pill, or granulate oral dosage form. An effective amount of up to about 5 wt % of a lubricant is included, and, optionally, about 5-10 wt % of a dispersing agent (“dispersant”). The core does not include a substantial amount of a redox-active metal salt.

The tablet coating, which covers the core, does not comprise a substantial amount of a redox-active metal salt such as a transition metal salt, for example, the coating does not contain titanium oxide or iron oxide. It has surprisingly been found that redox-active metal salts like titanium oxide and iron oxide can bring about the decomposition of picoplatin in formulations such as those of the present dosage form in vivo or in vitro. Therefore, the inventive oral dosage form excludes such materials, particularly from the coating, where such materials have been disposed to attenuate incident light. Rather, the coating of the invention can comprise less redox-reactive metal salts such as calcium sulfate as an opaquifying material, and can comprise additional materials that absorb or reflect incident light, provided that the additional material likewise is compatible with maintaining the picoplatin in bioactive, essentially pure form. Calcium sulfate does not bring about decomposition of the picoplatin, and calcium sulfate preferably in finely dispersed form within the coating serves to protect the picoplatin of the dosage form from photo-decomposition.

The invention also provides an oral dosage form for picoplatin prepared by a process comprising: (a) compressing a powder formed from a granulate comprising about 10-60 wt % picoplatin, wherein the picoplatin is a particulate of less than about 10 microns average particle diameter, about 40-80 wt % of a filler comprising a substantially water-soluble, water-dispersible or water absorbing carbohydrate, an effective amount of up to about 5 wt % lubricant, and optionally a dispersing agent to yield a tablet core, and (b) coating the tablet core to yield a coated tablet having a water-soluble or water dispersible coating on the outer surface thereof, wherein the core and the coating are substantially free of redox-active metal salts.

The invention also provides an oral dosage form for picoplatin prepared by a process comprising: (a) molding a powder formed from a granulate comprising about 10-60 wt % picoplatin, wherein the picoplatin is a particulate of less than about 10 microns average particle diameter, about 40-80 wt % of a filler comprising a substantially water-soluble, water-dispersible or water absorbing carbohydrate, an effective amount of up to about 5 wt % lubricant, and optionally a dispersing agent to yield a pill core, and (b) coating the pill core to yield a coated pill having a water-soluble or water dispersible coating on the outer surface thereof, wherein the core and the coating are substantially free of redox-active metal salts.

The invention also provides an oral dosage form for picoplatin prepared by a process comprising enclosing a plurality of granules comprising about 10-60 wt % picoplatin, wherein the picoplatin is a particulate of less than about 10 microns average particle diameter, about 40-80 wt % of a filler comprising a substantially water-soluble, water-dispersible or water absorbing carbohydrate an effective amount of up to about 5 wt % lubricant, and optionally a dispersing agent, in a gelatin capsule shell to yield encapsulated granules.

The invention also provides an oral dosage form for picoplatin prepared by a process comprising: (a) compressing a powder formed from a granulate comprising about 10-60 wt % picoplatin, wherein the picoplatin is a particulate of less than about 10 microns average particle diameter, about 40-80 wt % of a filler comprising a substantially water-soluble, water-dispersible or water absorbing carbohydrate, an effective amount of up to about 5 wt % lubricant, and optionally a dispersing agent to yield a tablet core which is substantially free of redox-active metal salts, and (b) coating the tablet core with gelatin to yield a geltab.

The present invention also provides a process for preparing an oral dosage form for picoplatin wherein the dosage form comprises a coating and a core, the core comprising a substantially dry powder comprising about 10 to 60 wt % picoplatin wherein the picoplatin is a particulate of less than about 10 microns average particle diameter, about 40-80 wt % of a filler comprising a substantially water-soluble, water-dispersible, or water-absorbing carbohydrate, and an effective amount of up to about 5 wt % of a lubricant; and optionally, about 5-10 wt % of a dispersant. The core is substantially free of redox-active metal salts. The core can be compacted or molded from a powder granulate, or can comprise a coated granulate. The core is formed by compacting a powder mixture comprising the picoplatin particulate, the filler and the lubricant, and optionally the dispersant; then, the compacted core is coated with a coating material free of redox-active metal salts, such that a continuous coating substantially completely covering the core is obtained. The coating comprises a substantially water-soluble or water-dispersible solid. The coating material can include gelatin, hard or soft, or can include a polymer or a sugar, or any other material suitable for human consumption. The coating serves to contain the compacted powder core, to prevent, or minimize the amount of dust released by handling of the tablet, and can protect the core from moisture and/or from light. It is advantageous to contain the cytotoxic picoplatin in the core with a coating such that dust is not produced, which could expose a person to inhalation or ingestion of the toxic material.

The coating, which does not include a redox-active metal salt such as a transition metal salt, such as the common opacifiers and/or colorants, titanium oxide or iron oxide, can comprise calcium sulfate and can include additional light-screening materials, excluding redox-active metal salts, that do not bring about picoplatin decomposition and are compatible-with picoplatin and the other core ingredients. The coating can also be substantially impermeable to water and/or to oxygen. The process of producing the tablet can also be carried out under subdued illumination to reduce photo-decomposition of the picoplatin.

The invention also provides a method of treating cancer in a human afflicted therewith, comprising orally administering a solid dosage form, including the oral dosage form of the invention or the oral dosage form prepared by the process of the invention, in a total dosage, at a frequency, and over a period of time adequate to provide a beneficial effect to the human. Other solid dosage forms of the invention, preferably with drug-compatible composite coatings, include coated pills, tablets, sachets and the like, which can comprise a plurality of coatings, e.g., a first coating on the core, covered by one or more overcoatings, e.g., to form geltab type dosage forms. Typically, total picoplatin doses are about 50-400 mg per administration, and the drug is administered to the mammal at intervals of about every day for at least two days, to intervals of every 6 weeks. The picoplatin administration can be accompanied by anti-emetic therapy; such as use of a corticosteroid such as dexamethaspne, a 5-HT3inhibitor such as palonosetron or ondansetron, a tranquilizer such as lorazepam, or any combination thereof.

Orally-ingestible dosage forms, such as the present coated solid cores, e.g., tablets or particulates, have a number of advantages over liquid dosage forms, such as intravenous solutions. An oral dosage form of picoplatin at different strengths permits physicians to easily titrate picoplatin to individual patients in response to observed side effects or to increase or decrease the dose to optimize efficacy or therapeutic index. This can be advantageous when picoplatin is given as a single agent or when it is used in combination with other anti-cancer agents, therapeutic agents or adjuvants. Other advantages of an orally ingestible form of picoplatin include ease of administration, convenience to patients, increasing patient compliance, and overall reduction in health care costs.

### DETAILED DESCRIPTION OF THE INVENTION

### Definitions

“Picoplatin” refers to cis-amminedichloro(2-methylpyridine)platinum(II), or [SP-4-3]-ammine(dichloro)(2-methylpyridine)platinum(II) as the drug is also termed, the structure of which is shown above. It is a compound belonging to the general class of redox-active metal complexes, in this case a complex of the third-row transition element platinum, the platinum being in the +2 oxidation state.

An “oral dosage form” as used herein refers to a physical dosage form that is adapted for oral administration, e.g., ingestion, wherein the form provides a preselected dose per individual, adapted to provide for a complete and rapid release of the drug in vivo after administration of the dosage form. In accordance with the present invention, the oral dosage form includes a core and a coating. The core includes a substantially dry powder including the picoplatin particulate of less than about 10 microns average particle diameter, a filler, and a lubricant.

The core can be formed by compaction, molding, or can be a granulated material. The average particle diameter of the picoplatin particulate can also be less than about 7 microns. The distribution of average particle diameters in the picoplatin particulate can be such that about 90% of the particles have individual particle diameters of less than about 5 microns. By “average particle diameter” is meant a number average particle diameter, as is well known in the art. The core is surrounded by a coating that covers the core, and serves to contain the materials of the core during storage and oral ingestion, as well as to protect the picoplatin contained in the core from various degradative agents such as light and oxygen. The core and the coating(s) (“the dosage form”) preferably do not contain any substantial amounts of redox-active metal salts such as transition metal salts. By a “granulate” is meant a divided form of a solid material formed of a plurality of individual granules of an intermediate coarseness, less fine than a powder, but not a monolith.

The oral dosage form is substantially water soluble, being adapted for oral administration. By “substantially water-soluble” is meant that the dosage form is sufficiently water-soluble to allow it to dissolve or disperse within the gastro-intestinal (GI) tract of the patient, so that the active ingredient of the formulation, picoplatin, can be absorbed into the patient's bloodstream through the mucosa of the GI tract. Thus, dissolution takes place within the period of time of a typical residence of an ingested substance within the GI tract, for example, within a period of time of several hours, preferably within a period of time of less than about 30 minutes, more preferably within a few minutes after ingestion of the tablet by the patient. However, although rapid dissolution is usually preferred, the dosage form can be further coated or otherwise adapted to permit controlled or prolonged release of the picoplatin, if desired.

The dosage form can be a coated tablet. By a “coated tablet” is meant herein a dosage form with a compacted powder core and a coherent coating, either hard or soft in texture, covering the core. The coating can be a plastic material. A “plastic” material, as the term is used herein, is a relatively malleable solid material that has sufficient rigidity to maintain a shape once formed, but which can be molded under pressure, for example, soft gelatin. The coating can be a water-soluble or water-dispersible substance that can be molded under pressure or applied as a viscous solution then subsequently dried, for example gelatin, a synthetic polymer, for example polyvinyl alcohol, or semi-synthetic polymer, for example hydroxypropyl methyl cellulose. The coating can be a layer of a water soluble solid such as a sugar, for example sucrose, that forms a sufficiently viscous solution to allow coating of the core with the viscous solution followed by drying of the coating that is thus applied.

The dosage form can be a geltab. A “geltab” as the term is used herein refers to tile dosage form comprising a coating, which can be soft gelatin or another soft, pliable gel-like material surrounding the compacted powder core. Such a coating is also preferably free of redox-active metal salts.

The dosage form can be a coated pill. A “pill” as the term is used herein is a molded but not compressed core wherein a binder assists in holding the picoplatin particulate and other components in a coherent mass. The dosage form can also be a sachet, wherein particulates are granulated and the granulations are coated individually or in small numbers, wherein pluralities of the particulates can be enclosed in packaging and then administered to provide the total dosage.

A “light-attenuating” coating, as the term is used herein, refers to a coating layer that covers the tablet that is adapted or treated so as to attenuate the intensity of light transmitted by the coating to the core containing the picoplatin. A coating may be light-attenuating without completely blocking or reflecting all incident light within the meaning herein. An “opaque” coating blocks or reflects most incident light. An “opaquifying” (or “opacifying”) agent is a material or a structure that serves to attenuate, reflect, disperse or absorb incident light such that the intensity of the light passing through the material containing the opaquifying agent is reduced compared to the intensity of the incident light. A light-attenuating coating is desirable due to the possible photo-decomposition of picoplatin, even in the solid form. An example of a light-attenuating coating is a coating comprising calcium sulfate, for example, a coating formed of a plasticized hydroxylpropyl methyl cellulose such as OPADRY® containing dispersed, solid calcium sulfate. Other salts, such as magnesium sulfate, can be used, provided that no redox-active metal salts such as transition metal salts are included.

A process is carried out under “subdued illumination,” as defined herein, when light intensities lower than the light intensities commonly used in manufacturing facilities, i.e., illuminations of an intensity sufficient to read written text, are used. Subdued light can also refer to light of spectral distribution known as “safe-light,” that is, light consisting predominantly of frequencies in the red range of the spectrum, where the picoplatin light absorption is less intense on a molar basis. Due to the potential light sensitivity of picoplatin, subdued illumination during practice of the process of the invention, along with the use of a substantially light-attenuating tablet coating, serve to enhance the stability and preserve high purity of the picoplatin in the oral dosage form.

A “core” as the term is used herein refers to a powder that can be derived from a coarser granulate that can compacted in the final dosage form, or can be molded, or can be used as a granulate that comprises picoplatin as a particulate of less than about 10 microns average particle size. The core further includes a filler comprising a carbohydrate and a lubricant, as the terms are defined herein. The core may also include other ingredients, such as a dispersant/disintegrant, an antioxidant, a buffer, a colorant, and the like. The core is preferably free of any redox-active metals or metal salts.

The core is covered by the coating, which is free of redox-active metal salts, to provide the oral dosage form of the invention. A “coating” as the term is used herein refers to a water-soluble or water-dispersible solid that is suitable for covering and sealing the core. Examples, such as are discussed above, include the coatings of a coated tablet, pill, granulation, or geltab. The coating can include ingredients such as an opaquifying material, for example calcium sulfate, an antioxidant, a colorant, a flavorant, and the like. The coating can also include imprints or embossed characters such as letters, numbers or symbols that are visually apparent and convey useful information to a care provider or a patient, such as the amount of the active ingredient picoplatin in the dosage form. There can also be a second coating on the outer surface of the first coating. The second coating is also preferably free of redox-active metal salts.

By a “substantially dry” material is meant a solid substance to which no exogenous water has been added and which has a relatively low wt % of contained water, typically less than about 5 wt %, preferably less than about 1-3 wt % of water, more preferably less than about 1 wt % of water. A substantially dry material need not be absolutely anhydrous within the meaning assigned herein, but the amount of residual water present in the material is limited. For example, lactose monohydrate, which includes 5 wt % water, can be used as a carbohydrate in the dosage form.

A “redox-active metal salt” as the term is used herein refers to salts of metals that can enter into redox reactions with picoplatin and includes transition metal salts such as Fe⁺³, but excludes the salts of Group 1 and Group 2 metals, i.e., alkali and alkaline earth metals such as Na, K, Mg, Ca, and the like.

A “transition metal salt” as the term is used herein, refers to salts of transition metals such as titanium, iron, copper, zinc and the like. The term does not encompass salts of aluminum or silicon. The term specifically includes oxides of transition metals, such as titanium oxide and iron oxide. It is recognized that picoplatin itself is generally not understood to be a transition metal salt, and, as the active pharmaceutical ingredient of the oral dosage form of the invention, picoplatin is not excluded from the dosage form. The terms “transition metal salt” and “redox-active metal salt” as used herein specifically exclude picoplatin and/or Pt-containing manufacturing impurities or degradation products derived from picoplatin. “Substantially free of a redox-active metal salt” means that the coating and/or core do not contain an amount of a redox-active metal salt, for example a transition metal salt, that can degrade the picoplatin, e.g., can reduce its bioactivity.

By “substantially free” of a redox-active metal salt or transition metal salt is meant that the coating or core has levels of one or more of these salts that do not, singly or in combination, significantly contribute to the degradation of the picoplatin, either in vitro e.g., in storage, or in vivo, e.g., after ingestion. Usually such amounts are no more than the total of the trace amounts of such salts normally present in adjuvants formulated or prepared so as to exclude them entirely.

By a “powder” is meant a material in the physical form of a solid that is divided into relatively fine particles. A powder can be a milled powder. A specific, example of a type of a powder is a micronized powder, that is, a powder whose constituent particles are of no more than about 10 microns in diameter. Such powders can be made by grinding coarser powders or solid masses to the desired fineness. A preferred method of forming a micronized powder is by jet milling. The powder material that forms the tablet core contains the picoplatin particulate, a fine powder of less than 10 microns average particle diameter, in combination with, or incorporated within, coarser powders such as carbohydrates, which can be of sufficient fineness to pass a 20-mesh or a 30-mesh screen, but which need not be of less than 10 microns average particle diameter.

By a “particulate,” in the context of the physical form of solid picoplatin disclosed herein, is meant a very fine powder wherein the average picoplatin particle diameter is less than about 10 microns, preferably less than about 7 microns, and preferably wherein at least about 90% of the individual picoplatin particles in a sample of the picoplatin have individual particle diameters of less than about 5 microns. The finely particulate nature of the picoplatin aids in its rapid and complete dissolution in the patient's GI tract. The picoplatin particulate can be a micronized material, a microcrystalline material, a-lyophilized material, or any combination thereof.

A “micronized” material is a powder wherein the majority of the particles making up-the powder have a particle diameter of about 10 microns or less. Preferably, the average particle diameter is less than about 7 microns. Particle diameters can range down to about 1 micron or less. A micronized solid can be crystalline or amorphous.

A “microcrystalline” material is a fine particulate wherein the solid is in crystalline form, the crystallites being predominantly of the specified dimensions. A microcrystalline material, as is known in the art, can be prepared by precipitation of the material from a solvent, such as by addition of a second liquid material in which the material is insoluble.

A “lyophilized” material is a solid that has been obtained by a step of lyophilization of a solution of the material. Lyophilization, as is well known, involves the vacuum sublimation of a solvent such as water and/or organic solvent from a frozen solution of the material, such that once the water and/or organic solvent is completely removed, a finely powdered solid material remains.

A “compacted” powder as can form the core of the dosage form is a powder that has been subjected to sufficient pressure to compress the powder, thereby removing air or optionally an inert gas from between the individual powder particles and causing the particles to fuse or adhere to each other. The particles adhere to each other with sufficient strength to allow at least the limited amount of handling needed to subsequently apply the coating material. A binder may be present in the powder to assist in the particles adhering to each other in the formation of the core. Alternatively a carbohydrate of the filler may act as a binder. Upon dissolution of the dosage form, for example in the GI tract of a patient, the particles disperse and dissolve.

A “molded” powder as can form the core of the dosage form is a powder that has been assembled into a cohesive mass without compression, such as by use of a binder that serves to cause adhesion of the powder. A “granulation” as can form the core of the dosage form is a particulate of relatively small size wherein each particulate can be covered with the coating to provide a plurality of coated granulated particles, such as forms a sachet.

A “filler” as the term is used herein refers to a water-soluble or water-dispersible solid composition comprising a carbohydrate. The core of the oral dosage form can include about 40-80 wt % of a filler. The filler serves to disperse the particulate picoplatin, inhibiting clumping of the sub-10 micron picoplatin particles, stabilizing the picoplatin chemically, and assisting in the dispersion or dissolution of the picoplatin in an aqueous medium. The bulk of the core in addition to the picoplatin, the lubricant, and the dispersant, if any, is generally provided by the filler, although additional ingredients can be present in the core.

A “carbohydrate” as the term is used herein includes a monomeric, dimeric, oligomeric or polymeric sugar derivative, such as glucose, fructose, lactose, sucrose, ribose, celluloses, modified celluloses (e.g., cellulose ethers, etc.), and the like. A carbohydrate molecule comprises carbon, hydrogen and oxygen, in an approximate molar ratio of 1:2:1. However, molecules deviating from this formula, such as deoxysugars and their oligomers/polymers, are also included within the term “carbohydrate” as used herein, provided sufficient hydroxyl groups are present to confer water-solubility or water-absorbability upon the substance. A carbohydrate may also contain other elements such as sulfur (e.g., sugar sulfonic acids) and phosphorus (e.g., sugar phosphates), without departing from the principles of the invention.

By a “substantially water-soluble” carbohydrate is meant that the carbohydrate is sufficiently water-soluble to allow it to dissolve in the aqueous environment of the gastrointestinal (GI) tract within a few hours, preferably within a few minutes. An example of a substantially water-soluble carbohydrate is a monosaccharide, for example glucose.

By a “substantially water-dispersible carbohydrate”is meant a carbohydrate that, while it may not totally dissolve in water, is nevertheless of sufficient hydrophilic nature that it freely disperses in water.

By a “substantially water-absorbing” carbohydrate is meant that the carbohydrate, although it does not completely or even to any significant degree dissolve in water, it nevertheless takes up, adsorbs, or absorbs water within its physical structure. For example, cellulose, such as microcrystalline cellulose, does not dissolve in water, but it becomes hydrated in the presence of water, absorbing several times its weight in water. This absorption of water by, for example, cellulose, can assist in the dissolution of the picoplatin; it is believed that this absorption of water by the water-absorbing carbohydrate acts to assist in the dissolution of the picoplatin within the GI tract by holding water molecules within close physical proximity to the surfaces of the finely particulate picoplatin.

By the term “cellulose” is meant herein a polymeric carbohydrate material made up mostly of a linear polymer of β(1-4)-linked D-glucose units. Cellulose is typically derived from a natural source such as wood pulp, cotton, or bacteria. Cellulose may be ground or comminuted, to create a finely particulate material. Alternatively, microcrystalline cellulose, such as is sold under the trademark Avicel®, can be used. For example, the Avicel® can be Avicel PH101®. By microcrystalline cellulose is meant a cellulose which has been subjected to partial acid hydrolysis, which serves to predominantly hydrolyze the amorphous regions of a sample of cellulose, leaving the more crystalline domains intact. Microcrystalline cellulose takes the physical form of a fine powder.

The term “modified cellulose” as used herein refers to a chemically or biologically modified cellulose. For example, sodium carboxymethyl cellulose, that is, cellulose that bears pendant carbbxymethyl groups as sodium salts, as is well known in the art, is a modified cellulose within the meaning herein. Likewise, methyl cellulose, ethyl cellulose, hydroxyethyl cellulose, hydroxypropyl cellulose, and methyl hydroxypropyl cellulose are modified celluloses within the meaning assigned herein. A cross-linked sodium carboxymethyl cellulose, also known as “croscarmellose sodium,” is a cross-linked, modified cellulose within the meaning herein. Croscarmellose sodium is a dispersant or disintegrant within the meaning of the terms herein.

A “lubricant” or “glidant” within the meaning herein is a substance that serves to coat the surface of particles and reduce the friction of inter-particle movement, such as during powder handling operations, for example, when forming the core for the tablets. Reducing the friction serves to reduce static electricity buildup and particle clumping or aggregation, for example during the milling, powder handling, and compression processes typically used to produce the oral dosage form of the invention.

A “dispersant” or “disintegrant” is a substance that can be a component of the dosage form of the invention that aids in the dispersion of the tablet core upon exposure to an aqueous medium, for example within the GI tract of a patient. It is believed that dispersants act to increase the solvation of the surfaces of solid picoplatin particles within the aqueous medium, thereby reducing particle-particle adhesion and clumping while aiding in dissolution of the solid through improved surface wetting. Examples of dispersant include croscarmellose sodium and povidone. Povidone, also known as poly(vinylpyrrolidone), is a polymeric material bearing multiple pyrrolidone units along a poly(vinyl) backbone.

The coating of the dosage form can include a polymer, such as a modified cellulose. Preferred modified cellulose materials include cellulose ethers, such as hydroxypropyl methyl cellulose. A commercial formulation adapted for coating containing hydroxypropyl methyl cellulose is OPADRY®, sold by Colorcon, Inc. of 1936 West Point Pike, West Point, Pa. 19486.

The coating of the dosage form can also include a gelatin, such as a soft gelatin or a hard gelatin. “Gelatin,” as the term is used herein, is a collagen-derived material that is about 98-99% protein by dry weight. The approximate amino acid composition of gelatin is: glycine 21%, proline 12%, hydroxyproline 12%, glutamate 10%, alanine 9%, arginine 8%, aspartate 6%, lysine 4 %, serine 4%, leucine 3%, valine 2%, phenylalanine 2%, threonine 2%, isoleucine 1%, hydroxylysine 1%, methionine and histidine 1%,and tyrosine <0.5%.

The coating of the dosage form can also include a sugar, for example: sucrose, as is well known in the art. See, for example, Remington, The Science and Practice of Pharmacy, 21^{st} ed., 2006, Lippincott Williams and Wilkins, which is incorporated herein by reference in its entirety, Chapter 45, “Oral Solid Dosage Forms” by Edward M. Rudnic, Ph.D. and Joseph B. Schwartz, Ph.D.

The coating of the dosage form can also include a synthetic polymer, such as polyvinyl alcohol (PVA), which can be prepared by hydrolysis of polyvinyl acetate as is well known in the art. An example of PVA is Elvanol®, a product of DuPont, Inc.

The present invention provides an oral dosage form for picoplatin wherein the dosage form is a coated tablet comprising a coating and a core, the core comprising a substantially dry powder comprising about 10 to 60 wt % picoplatin, preferably about 15-40 wt %, wherein the picoplatin is a particulate of less than about 10 microns average particle diameter, about 40-80 wt % of a substantially water-soluble, water-dispersible, or water-absorbing carbohydrate, and an effective amount of up to about 5 wt % of a lubricant; wherein the dosage form is substantially free of redox-active metal salts other than the picoplatin itself.

The dosage form, which is adapted for oral administration of picoplatin; includes a coating, which is formed of a substantially water-soluble material. The coating is sufficiently water-soluble to allow it to dissolve in the gastro-intestinal (GI) tract of the patient, so that the formulation is released for dissolution and absorption into the patient's blood stream through the mucosa of the GI tract. The coating does not contain any redox-active metal salt, although it is understood that it may contain picoplatin in trace amounts such as may result from contamination of the coating material with the core picoplatin material, such as may occur during the coating process.

Any non-toxic water-soluble material of sufficient physical strength and film-forming capacity that is suitable for human consumption can be used to form the coating, provided that it is compatible with the picoplatin and the other ingredients making up the core. The term “compatible” includes compatibility of the coating material with the core material during manufacture, during storage, and post-administration of the dosage form. The term encompasses chemical compatibility, i.e., a lack of molecular degradation of the picoplatin or any of the other core ingredients brought about by a component of the coating material. The term also encompasses biochemical compatibility, i.e., a lack of interference of coating components with the desirable attributes of rapid and complete dissolution of the picoplatin after administration of the dosage form and of the bioavailability of the picoplatin released from the dosage form post-administration.

For examples the coating material can include gelatin, such as a hard gelatin or a soli gelatin, as are well known in the art. “Gelatin,” as the term is used herein, is a collagen-derived material that is about 98-99% protein by dry weight. The approximate amino acid composition of gelatin is: glycine 21%, proline 12%, hydroxyproline 12%, glutamate 10%, alanine 9%, arginine 8%, aspartate 6%, lysine 4%, serine 4%, leucine 3%, valine 2%, phenylalanine 2%, threonine 2%, isoleucine 1%, hydroxylysine 1%, methionine and histidine <1%, and tyrosine <0.5%. Hard gelatin and soft gelatin differ in their texture or consistency; soft gelatin is more malleable and gel-like than is hard gelatin.

The coating material can include a sugar, for instance sucrose, which can form a hard, smooth coating around the powder of the tablet core. Sugar-coated tablets, such as are well-known in the art, are described in detail in Remington, The Science and Practice of Pharmacy, 21^{st} ed., 2006, Lippincott Williams and Wilkins, which is incorporated herein by reference in its entirety. A sugar coating can contain additional materials such as plasticizers, anti-oxidants, colorants, flavorants, and the like.

The coating material can include a polymer, such as a modified cellulose, which is a preferred material for coating the tablet core. For example, modified celluloses such as hydroxypropyl methyl cellulose, or methyl cellulose, or hydroxypropyl cellulose, can be used in the tablet coating. An example of a modified cellulose suitable for use in the coating material is OPADRY®, a form of hydroxypropyl methyl cellulose manufactured by Colorcon Inc. (www.colorcon.com). The coating can also include a plasticizer for the polymer, if needed. An example of a plasticizer is polyethyleneglycol. The coating can also include an anti-foaming agent such as Antifoam C Emulsion.

The tablet coating can be adapted to attenuate the exposure of the contained picoplatin to any incident light as may fall on the tablet. Due to the known instability of picoplatin to light, even in the solid form, attenuation of incident light serves to maintain the purity of the picoplatin contained within the tablet. The coating can attenuate incident light to a significant degree, preferably by at least about 50% at typical room illumination levels, or at least by about 75%, or at least by about 90%, or at least by about 95%. This can be accomplished by incorporation into the coating material of an opaquifying agent, not including a redox-active metal salt, for example, a light-reflecting substance such as finely dispersed CaSO₄ can be incorporated. An example of a suitable opaquified coating material is OPADRY° formulated to contain a dispersed solid form of CaSO₄. The opaquifying agent serves to attenuate incident light by absorbing or reflecting the light. Other opaquifying agents not including redox-active metal salts can be included. Agents such as titanium oxide and iron oxide are specifically excluded from use as an opaquifying agent in the coating. The coating can also include a colorant, provided that the colorant does not contain a redox-active metal salt, which both can serve to reduce the light exposure of the picoplatin within the tablet and to help identify the tablet to care providers and patients.

The dosage unit core includes a powder comprising picoplatin as a particulate of less than about 10 microns average particle diameter, a carbohydrate, and a lubricant. Multiple types of carbohydrate, lubricant, or both, can be present. Additional ingredients can also be present in the core, such as a dispersant, which can serve to disperse the particles of the picoplatin-containing powder in the patient's GI tract. Other ingredients that can be present include stabilizers such as binders, anti-oxidants, buffers, colorants, or other medicaments, including other anti-cancer drugs.

As mentioned above, picoplatin is a tetracoordinate platinum(II) complex, and such complexes are known to possess certain instabilities that the unit dosage form of the invention is adapted to avoid or minimize. For example, tetracoordinate platinum(TI) complexes, as described above, are susceptible to addition of molecular chlorine. Molecular chlorine can be formed in situ when chloride (a halide) and an oxidizing reagent (such as atmospheric molecular oxygen) are present. Thus, chlorides are preferably excluded from the formulation. Solid oxidizing agents that can be used, for example, as microbiocides, including chlorite and povidone iodine, are preferably excluded from the formulation. Also, since compounds comprising moieties including ═NH, —NH2, and —SH, as can be found in various excipients such as dispersant/disintegrants, can react with tetracoordinate platinum(II) complexes like picoplatin, either in situ or in vivo, the dosage form preferably does not include any such compounds, for example as functional groups of dispersants or colorants.

Due to the instability of picoplatin in the presence of redox-active metal salts, such as iron oxide and titanium oxide, redox-active metal salts are excluded from the oral dosage form. For this reason, redox-active metal salts are not included in the dosage form, either in the core or in the coating, with the understanding that picoplatin itself, although it could itself be viewed as a redox-active metal salt, is not within this exclusion. In particular, the coating does not include a metal oxide like titanium oxide as an opaquifying agent, but rather uses other materials such as calcium sulfate, which can be finely dispersed to provide a reflective coating. It has surprisingly been found that titanium oxide, for example when incorporated into a well known coating material such as hydroxypropyl methyl cellulose (e.g., OPADRY®), can bring about the degradation of picoplatin to materials such as 2-picoline and trichioroaminoplatinate, as is shown in the Examples. Therefore the coating material of the invention does not incorporate titanium oxide. For light attenuation, either an inorganic or an organic component can be included in the coating material. Calcium sulfate in finely ground form has been found to confer opacity on the coating, and does not bring about picoplatin degradation. Suitable colorants can likewise be used for light attenuation in the tablet coating.

While the solid picoplatin in the tablet may be only moderately susceptible to degradation in the presence of reactive functional groups such as amino groups, due to the relatively unreactive nature of solid materials, during processes such as in the compounding of the formulation, and particularly during the process of dissolution in the stomach, the absence of reactive ingredients can assist in maintaining picoplatin purity. In the microenvironment that exists as the solid material is first released into the stomach acid, local high concentrations of dosage form ingredients exist in close physical proximity to the surfaces of the dissolving picoplatin particulates. It may take several minutes, if not longer, for these small particulates to pass completely into solution, and during that time the ingredients of the formulation other than the picoplatin that are likewise dissolving are present in solution in high local concentrations adjacent to the dissolving picoplatin particles. The absence of picoplatin-reactive functional groups on substances that can exist in locally high concentrations in these stomach microenvironments is therefore advantageous.

The picoplatin that is contained in the powder of the core is a particulate of an average particle diameter of less than about 10 microns. The picoplatin particulate can be a micronized material, a microcrystalline material, a lyophilized material, or any combination thereof. The picoplatin can be milled or micronized by jet milling, or by any other process that can provide micronized powders of suitably small average particle diameters. Micronized picoplatin, due to tire favorable surface area to mass ratio that results from the presence of fine particles, aids in the rapid and complete dissolution of an effective amount of the picoplatin in the patient's GI tract after administration of the dosage form. Micronized picoplatin can be composed of crystalline or amorphous solid picoplatin.

The picoplatin particulate can also be a microcrystalline solid, wherein the powder is composed of crystals of appropriately small physical dimension. Microcrystalline materials can be formed, as is known in the art, by precipitation of a solid from a solution by addition of a liquid in which the material is insoluble, for example with high shear or agitation.

The picoplatin particulate can also be a lyophilized powder, such as is formed by lyophilization of a solution of the picoplatin. The picoplatin particulate can also have been formed by any combination of the above-listed methods of forming fine particulates; for example, a microcrystalline material can be micronized such as by jet milling to reduce particle size, or a material that has been recovered from an aqueous solution by lyophilization can be micronized, and so forth.

The mixture of the picoplatin, the carbohydrate, the lubricant, and any other ingredients that may be present is also in form of a powder, but is not as fine a powder as the picoplatin particulate. The powder can be a mixture of picoplatin particulates and particles of the other ingredients, or, preferably, the particles making up the powder can have incorporated within substantially every one of them a plurality of the fine textured picoplatin particulates dispersed within particles of the other components such as the carbohydrate. It is preferred that the mixture be an intimate mixture, where picoplatin particulates are closely mixed with the additional ingredients of the formulation, as the greater is the surface area of the component picoplatin particles, and the more intimately these picoplatin particles are mixed with the carbohydrate and with the optional dispersant or disintegrant, the more rapidly and completely the picoplatin will dissolve or disperse after administration of the tablet to the patient. Rapid and complete dissolution of the picoplatin is generally desirable in terms of providing a maximally effective treatment to the patient.

The powder of the core is in a substantially dry form; the water content of ingredients such as carbohydrates and dispersants is controlled to minimize the wt % of water in the formulation. Water, under some conditions, can react with picoplatin, resulting in decomposition. Therefore, the water content of the dosage form is preferably limited to less than 5 wt %, preferably less than 1-3 wt %, and more preferably to less than 1 wt % of the composition. It is understood that certain carbohydrates, for example lactose, may exist in the form of a hydrate, such as a monohydrate which contains 5 wt % water; such hydrates may be used, but exogenous water is preferably excluded as much as is practicable.

The picoplatin, which makes up at least about 10 wt % of the core and can make up to about 60 wt % of the core, is preferably anhydrous, and is handled under conditions during the formulation processes to maintain its dry state, Dryness can be maintained through use of suitable engineering controls, such as operation under a dry, inert atmosphere, as is well known in the art.

The filler, which comprises about 40-80 wt % of the core, comprises a carbohydrate. Suitable carbohydrates can be selected from a group consisting of a monosaccharide, a disaccharide, a sugar alcohol, a cellulose, a modified cellulose and mixtures thereof. Carbohydrates are water-soluble, water-dispersible, or are water-absorbing, that is, the fillers either dissolve completely in water, freely disperse in water, or are sufficiently hydrophilic to absorb substantial amounts of water within their structure. For example, fructose is water-soluble, certain hemicelluloses are water-dispersible, and cellulose is water-absorbing. More than one carbohydrate can be present in the dosage form. The total carbohydrate is preferably present at about 40-80 wt % of the formulation.

An example of a monosaccharide is fructose. Other examples include without limitation glucose, xylose, mannose, galactose, ribose, and the like. Examples of a disaccharide include lactose and sucrose.

Examples of sugar alcohols include sorbitol, ribitol, mannitol and xylitol. An example of a hemicellulose is a wood-derived, alkali-soluble hemicellulose.

An example of a cellulose is microcrystalline cellulose. Another cellulose is a finely ground or comminuted cellulose, such as a high grade wood pulp cellulose that has been ground to a powder form.

An example of a modified cellulose is sodium carboxymethyl cellulose. Other examples include without limitation methyl cellulose, ethyl cellulose, hydroxyethyl cellulose, hydroxylpropyl cellulose, and methyl hydroxylpropyl cellulose. Some examples of modified cellulose are water soluble, whereas others are water-dispersible or water-absorbing.

The formulation of the invention includes a lubricant in an effective amount. A lubricant, for example the salt of a fatty acid, more specifically magnesium stearate, can serve as a processing aid in handling the powder of the core, in particular the sub-10micron picoplatin powder, by assisting in avoidance of particle clumping, such as during milling operations. A lubricant can be present at up to about 5 wt % of the core.

A dispersant, which serves to enhance the dispersal of the tablet core in an aqueous medium, such as in the GI tract of a patient, facilitates rapid dissolution. The dispersant tends to assist in dispersion of the particles when they first encounter the aqueous medium, thus helping to preserve the favorable surface area to mass ratio of the fine picoplatin powder. An example of a dispersant is cross-linked sodium carboxymethyl cellulose, also known as croscarmellose. Another example is povidone, also known as polyvidone, poly(vinylpyrrolidone), or PVP. The formulation can comprise about 5-10 wt % of the dispersant. More than one dispersant can be present in the dosage form.

The dosage form can include other ingredients, but does not include redox-active metal salts, and preferably does not include oxidants, or compounds comprising halo, ═NH, —NH₂, or —SH moieties. Other components preferably lacking such groups can be included. For example, anti-oxidants can be included. Examples include BHA or BHT. Colorants, such as food dyes, can be included.

Thus the ratio of picoplatin to carbohydrate filler to dispersant (if present) to lubricant is about 1:1.5-3.0:0.1-0.3:0.25-0.1. In one embodiment, the dosage form comprises about 200 mg of the core material, comprising about 50 mg of micronized picoplatin, about 116 mg of lactose, about 20 mg of microcrystalline cellulose, about 8 mg of croscarmellose sodium, about 4 mg of povidone, and about 2 mg of magnesium stearate, as an compacted admixture; covered by the coating. The coating can be opaquified, as by incorporation of CaSO₄ into the coating.

The invention further provides a process for preparing an oral dosage form for picoplatin wherein the dosage form comprises a coating and a core, the core comprising a substantially dry powder comprising about 20 to 55 wt % picoplatin wherein the picoplatin is a particulate of less than about 10 microns average particle diameter, about 40-80 st % of a filler comprising a substantially water-soluble, water-dispersible, or water-absorbing carbohydrate, and an effective amount of up to about 5 wt % of a lubricant; and wherein the dosage form is free of redox-active metal salts; the process comprising forming the core of a powder mixture of the picoplatin particulate, the carbohydrate and the lubricant, then, coating the core with a coating material such that a protective coating substantially completely covering the core is obtained.

The constituent materials used in the process of the invention are as described above for the unit dosage form of the invention. The process of the invention comprises preparing the powder of the formulation that is substantially dry. Then, the powder can be compacted or compressed into a tablet form suitable for applying the coating, then, coating the compacted core with a suitable coating material to provide a coaled tablet or geltab of the invention. Alternatively, the powder can be molded, as with the addition of a binder, to provide the core which is then coated as above. Or, the powder can be granulated, and the granulations individually coated with the coating.

As a non-limiting example of how the process of the invention can be carried out, lactose, microcrystalline cellulose, and a lubricant such as magnesium stearate can each be ground to pass a 20-mesh screen or a 30-mesh screen, then can be blended with the picoplatin particulate of less than 10 microns average particle diameter in a granulator. The picoplatin and the lactose can optionally be blended together prior to addition of any other ingredients. The picoplatin particulate can have been prepared earlier by a jet milling process, or by formation of microcrystals, or by lyophilization, or any combination of these processes that provides particulates of the requisite dimensions. A dispersant, for example povidone, such as in the form of a powder that passes a 20-mesh screen or a 30-mesh screen, can be added to the mixture in the granulator. Mixing of the solids then can take place, such as by using high-shear granulation, so as to form an admixture of the component materials. The admixture of the sparingly water-soluble picoplatin (having a solubility of less than 1 mg/mL, or about 0.1%; in water) with the water-soluble, water-dispersible or water-absorbing carbohydrate, the lubricant, and optionally with the dispersant, serves to enhance rapid and substantially complete dissolution of an effective amount of the picoplatin in the patient's GI tract. While the particles of the various materials can be merely mixed together to form a powder, it is preferable to mix them with sufficient thoroughness such that the picoplatin particulates are incorporated within or on the surface of the typically larger carbohydrate particulates. Insofar as possible, the presence of redox-active metals is excluded from the manufacturing process and equipment.

Following the milling and mixing processes, the formulation can be dried, for example, spread in a thin layer on a tray, which is then held under drying conditions. For examples the powder on the tray can be warmed to a moderate temperature, such as about 40-80° C., and held under a partial vacuum or in the presence of a drying agent, for example, P₂O₅. Residual water can be controlled such that the water content is less than 5 wt %, more preferably less than 1 -3 wt %, even more preferably less than 1 wt %, of the solid mixture.

Following drying, additional milling can take place. The bulk material can be sifted through the screen, if desired, to remove any larger particles that may be present. For example, a predominant portion of a sample of the mixed powder can pass through a 20-mesh screen. Preferably, the bulk of the powder can pass through a 30-mesh screen.

The powder can be kept substantially dry through the use of suitable engineering techniques and controls, such as storage under controlled atmosphere, interjection of suitable drying steps into the process tor preparation, and storage in the absence of atmospheric moisture. The powder can also be handled under subdued light, in order to minimize the amount of photolytic decomposition of picoplatin, which is well known to be light-unstable. The control of incident light can be carried out by suitable engineering controls, such as processing the material in opaque vessels, conveying it and drying it under cover or in the dark, or the use of safe-lights such as can be used for photographic processing, It is desirable to minimize incident light in carrying out the inventive process. Furthermore, contact with metals as can cause decomposition of the picoplatin during the process is preferably avoided.

After the final mixing, drying, and screening steps of the process are carried out, the powder is compacted and coated with the coating material. Compaction of the substantially dry powder that will make the tablet core can be carried out using any suitable method as is known in the art. For example, dry granulation compacting or direct compression can be used, wherein irreversible deformation of the powder bed takes place in a tablet press to provide a compacted core that is suitable for coating. The carbohydrate, for example lactose, can have cohesive properties and thus serve as a binder; therefore additional binders are not necessary, but can be added if desired. Compaction serves to compress the finely particulate picoplatin with the carbohydrate and lubricant, optionally with a dispersant, into a cohesive, but preferably not a completely fused, mass. Suitable compaction or compression techniques are described in Remington, The Science and Practice of Pharmacy, 21^{st} ed., 2006, Lippincott Williams and Wilkins, which is incorporated herein by reference in its entirety. See, for example, Chapter 45, “Oral Solid Dosage Forms” by Edward M. Rudnic, Ph.D. and Joseph B. Schwartz, Ph.D.

The compacted cores, or alternatively, molded or granulated cores, can then be coated with a suitable coating agent, as is well known in the art, which can include gelatin, a sugar, or a polymer, for example a modified cellulose such as hydroxypropyl methyl cellulose. See, for example. Chapter 46 of Remington, “Coating of Pharmaceutical Dosage Forms,” by Stuart C. Porter, Ph.D., which is incorporated herein by reference in its entirety. A preferred coaling material includes a modified cellulose, for example, hydroxypropyl methyl cellulose. The coating can be applied and dried by any suitable technique known in the art. As discussed above, the coating material can include an opaquifying agent such as CaSO₄, a colorant, an anti-oxidant, or the like. Incorporation of an anti-oxidant, such as BHA, BHT, or a tocopherol, into the coating material can be used to provide a barrier to oxygen penetration, which is desirable for the exclusion or reduction of the amount of oxygen, which can cause degradation of picoplatin.

The dosage form of the invention, or the dosage form prepared by the method of the invention, can have about a ±10% spread in the actual amount of contained picoplatin relative to the nominal composition. For example, a dosage form with a nominal 200 mg weight containing a nominal 50 mg of picoplatin, can have about 45 to 55 mg of picoplatin as measured for that individual sample. The dosage form has low and limited amounts of various impurities; for example it should contain no more than about 1% of each of several possible residual impurities, e.g., from the degradation, or manufacture of the picoplatin, such as potassium tetrachloroplatinate, picoline, trichloropicolineplatinate, or trichloroaminoplatinate.

The invention also provides a method of treating cancer in a human afflicted therewith, comprising orally administering a solid or liquid dosage form, including the oral dosage form of the invention or the oral dosage form prepared by the process of the invention, in a total dosage, at a frequency, and over a period of time adequate to provide a beneficial effect to the human. Other solid dosage forms of the invention, preferably with drug-compatible coatings, include as pills, tablets, sachets and the like. Typically, total picoplatin doses are about 50-400 mg per administration, and the drug is administered to the mammal at intervals of about every day for at least two days, to intervals of every 6 weeks. The frequency can be metronomic, that is, dosage forms can be administered, for example, daily for several days, then no dosage form administered for several days, then the sequence repeated multiple times. The picoplatin administration can be accompanied by anti-emetic therapy, such as use of a corticosteroid such as dexamethasone, a 5-HT3 inhibitor such as palonosetron or ondansetron, a tranquilizer such as lorazepam, or any combination thereof.

The dosage form is adapted to administered orally, and a typical oral dose includes a plurality of the tablets, for example, eight tablets containing 50 mg of picoplatin each can be used to provide a 400 mg total dose to the patient.

It is well known that picoplatin can be active against tumors that possess, or have developed, resistance to “first-generation” and “second generation” organoplatinum anti-cancer drugs such as cisplatin oxaliplatin and carboplatin. For example, the oral dosage form of the invention or prepared by the process of the invention can be used to treat patients with non-hematological malignancies, such as patients with solid malignant tumors, in particular, those patients whose solid tumors are cisplatin oxaliplatin or carboplatin refractory. Specific types of solid malignancies that can be treated with the oral dosage form of the invention, or with the oral dosage form prepared by the process of the invention, include without limitation, lung cancer, including small ceil lung cancer, non-small cell lung cancer, head and neck cancer, ovarian cancer, prostate cancer (e.g., HRPC), colorectal cancer, sarcomas, breast cancer, carcinoid tumors, and the like.

A plurality of the oral dosage forms of the invention or prepared by the process of the invention can be given to a patient as a single total dosage ranging from about 50 mg to 400 mg up to about 2000-3000 mg of picoplatin. The total dosage can be given in the form of a suitable number of the dosage forms, i.e., for a 200 mg dose of picoplatin, 4 unit dosage forms containing 50 mg each of picoplatin can be administered. For a larger dose, such as 2000 mg, larger unit dosage forms containing more picoplatin particulate can be used, for example, ten tablets each containing about 200 mg of picoplatin each can be used. Alternatively, a single dosage form can be given more frequently, for example, daily. It is preferred that the entire number of a plurality of the dosage forms for a given administration be administered within a short interval of time, for example, within a period of time of about five minutes. Thus, if a given administration includes a nominal 200 mg of the picoplatin, and nominal dosage forms containing 50 mg each of picoplatin are used, all four capsules should be administered to the patient within about five minutes. For dosage levels of 50- 400 mg total picoplatin, assuming a average patient body surface area of 1.7 m², these doses are equal to about 30 to 235 mg/m², respectively, wherein the m² refers to the body surface area of the patient, assuming 100% absorption, if absorption rates are less than 100%, oral doses can be correspondingly increased. This dosage can be repeated as medically indicated; for example, the dosage can be repeated daily, weekly, about every two weeks, about every three weeks, about every four weeks, about every five weeks, or about every six weeks, as is deemed medically indicated.

The solid dosage form of the invention is adapted for oral administration to a human wherein at least about 10% of the total picoplatin contained in the dosage form is bioavailable to the human following ingestion of the dosage form. Or, the solid dosage form of the invention is adapted for oral administration to a human wherein at least about 20%, or at least about 30%, or at least about 40%, or at least about 50%, of the total picoplatin contained in the dosage form is bioavailable to the human following oral ingestion of the dosage form by the human.

The picoplatin oral dosage form of the invention has up to about 40percent bioavailability, or up to about 50% bioavailability, following oral ingestion by a human. More specifically, the picoplatin oral dosage form of the invention has about 10-40%, or about 20-40%, or about 30-40% bioavailability in humans following oral ingestion. Alternatively, the picoplatin oral dosage form of the invention has about 30-50%, or about 40-50% bioavailability in humans following oral ingestion.

The method of treatment of the invention can further comprise administering an anti-emetic therapy to the patient, either within about 30 minutes prior to or, substantially concurrently with, administration of the inventive tablets. The anti-emetic therapy can include administration of a corticosteroid or a 5-HT3 receptor antagonist, or both. For example, the corticosteroid can be dexamethasone. The 5-HT3 receptor antagonist can be palenosetron or ondansetron. Additional anti-emetic agents can be administered, such a tranquilizer, for example, lorazepam.

The method of treatment of the invention can further include administering an additional medicament or radiation therapy with the unit dosage form or a plurality of the dosage forms of the invention or prepared by the method of the invention. The additional medicament can be an anti-cancer medicament. For example, an additional anti-cancer medicament can comprise, without limitation, a taxane, a taxol derivative, a growth factor receptor inhibitor, e.g., an anti-EGFR antibody, a Her2 inhibitor, a Vinca alkaloid derivative, a second orgahoplatinum compound, a nucleotide analog (e.g., 5-FU), a mustard agent, an alkylating agent or the like. Alternatively, the additional medicament can be selected to treat a complication of the cancer, such as ah infection, or to provide relief to a patient from a symptom of the cancer, such as a fever.

The invention further provides a kit comprising packaging containing a sufficient number of the dosage forms of the invention or prepared according to the method of the invention to provide for a course of treatment. A kit can further include instructional materials, such as instructions directing the dose or frequency of administration. For example, a kit can comprise sufficient daily doses for a prolonged period, such as a week, or can comprises multiple dosage forms for a single administration when the dose is to be repeated less frequently.

Certain examples are provided below in order to assist in understanding embodiments of the present invention; they should not, however, be considered as limiting the present invention, which are described in the claims.

### EXAMPLES

### Example 1

### Formation of Impurities from Picoplatin in Solutions Including TiO₂ vs. CaSO_{4 j}

Picoplatin solutions were mixed with solutions of TiO₂, clear OPADRY (no TiO₂) and standard coating OPADRY containing TiO₂ or CaSO₄. After standing, the solutions were analyzed by HPLC for picoplatin decomposition products 2-picoline and trichloroaminoeplatinate (TCAP). The results are shown in Table 1.

TABLE 1

| | 2-Picoline % | TCAP % |
|---|---|---|
| Control | 0.02 | 0.07 |
| TiO₂ | 0.06 | 0.15 |
| OPADRY (clear) | 0.02 | 0.09 |
| TiO₂ OPADRY | 0.24 | 0.83 |
| CaSO₄ OPADRY | 0.02 | 0.08 |

The CsSO₄ OPADRY coating was shown not to cause the degradation in the picoplatin observed for TiO₂ or for the TiO₂ OPADRY product.

### Example 2

### Effect of FeSO²⁺ Concentration on TCAP Formation from Picoplatin as a Function of Time

Solution of FeSO²⁺ were made up and added to solutions of picoplatin in water providing final Fe²⁺ concentrations as shown. At the designated time points, TCAP percentages as % conversion from picoplatin were determined by HPLC, shown in Table 2.

TABLE 2

| Fe²⁺ | | 100 | 20 | 5 | 2 | 0.4 | 0.2 |
|---|---|---|---|---|---|---|---|
| (ppm) | | | | | | | |
| Fe²⁺ | | 1.79 | 0.36 | 0.089 | 0.036 | 0.0071 | 0.0036 |
| (mM) | | | | | | | |
| 0 | hrs | 0.06 | 0.08 | 0.09 | 0.00 | 0.01 | 0.01 |
| 4.5 | hrs | | | | 0.13 | 0.02 | 0.00 |
| 8 | hrs | 0.80 | 0.79 | 0.60 | | | |
| 15 | hrs | | | | 0.24 | 0.09 | 0.03 |
| 1 | week | 2.57 | 2.38 | 0.63 | 0.30 | 0.13 | 0.04 |

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. An oral dosage form for picoplatin wherein the dosage form comprises a solid core comprising about 10 to 60 wt % particulate picoplatin wherein the picoplatin is a particulate of less than about 10 microns average particle diameter, about 40-80 wt % of a filler comprising a substantially water-soluble, water-dispersible, or water-absorbing carbohydrate, and an effective amount of up to about 5 wt % of a lubricant; and a continuous coating on the outer surface of the core; wherein the core and the coating are substantially free of a redoxactive metal salt.

2-68. (canceled)
